(19) United States
(12) Patent Application Publication
(10) Pub. No.: US 2023/0410581 A1
(43) Pub. Date: Dec. 21, 2023

(54) HEALTH PROMOTION SYSTEM AND HEALTH PROMOTION METHOD

(71) Applicant: TOYOTA JIDOSHA KABUSHIKI KAISHA, Toyota-shi (JP)

(72) Inventors: Issei NAKASHIMA, Tokyo-to (JP); Toru MIYAGAWA, Seto-shi (JP); Hideo HASEGAWA, Nagoya-shi (JP); Yusuke KINOSHITA, Tokyo-to (JP); Keisuke SUGA, Nagoya-shi (JP)

(73) Assignee: TOYOTA JIDOSHA KABUSHIKI KAISHA, Toyota-shi (JP)

(21) Appl. No.: 18/138,857

(22) Filed: Apr. 25, 2023

(30) Foreign Application Priority Data
Jun. 20, 2022 (JP) 2022-098868

(51) Int. Cl. G07C 9/00; G07C 9/37; G06F 21/32; G06F 21/44

(52) U.S. Cl. CPC G07C 9/00563; G07C 9/37; G06F 21/32; G06F 21/44

(57) ABSTRACT

A health promotion system using a computer includes: an operation mechanism configured to operate in response to an operation performed by a person; a control unit configured to control the operation mechanism; identification information acquisition unit that acquires identification information for identifying the person who operates the operation mechanism; and motion detection unit that detects a motion of the person. The control unit determines whether or not the motion detected by the motion detection unit satisfies a health promotion motion that is predetermined for the person identified by the identification information, and operates the operation mechanism in response to the operation performed by the person only when the detected motion satisfies the health promotion motion.

### CROSS REFERENCE TO RELATED APPLICATIONS

This application is based upon and claims the benefit of priority from Japanese patent application No. 2022-098868, filed on Jun. 20, 2022, the disclosure of which is incorporated herein in its entirety by reference.

### BACKGROUND

The present disclosure relates to a health promotion system and a health promotion method.

Japanese Unexamined Patent Application Publication No. 2010-140119 discloses a system that determines a user's intention or state by analyzing physical information of the user, and causes a robot to perform a predetermined motion corresponding to the user's intention or state. According to the motion performed by the robot, a user voluntarily maintains the motor functions of the user and activates the mental and physical activities of the user, whereby care prevention is promoted.

### SUMMARY

The system disclosed in Japanese Unexamined Patent Application Publication No. 2010-140119 does not significantly motivate a user to exercise, that is, it simply recommends exercise, and therefore the user may not exercise. That is, the system disclosed in Japanese Unexamined Patent Application Publication No. 2010-140119 has a problem that it is inferior in health promotion effect.

The present disclosure has been made in view of the above-described circumstances, and an object thereof is to provide a health promotion system and a health promotion method by which excellent health promotion effects can be obtained.

A first exemplary aspect is a health promotion system using a computer, the health promotion system including:
an operation mechanism configured to operate in response to an operation performed by a person;
a control unit configured to control the operation mechanism;
identification information acquisition unit that acquires identification information for identifying the person who operates the operation mechanism; and
motion detection unit that detects a motion of the person, in which the control unit determines whether or not the motion detected by the motion detection unit satisfies a health promotion motion that is predetermined for the person identified by the identification information, and the control unit operates the operation mechanism in response to the operation performed by the person only when the detected motion satisfies the health promotion motion.

Another exemplary aspect is a health promotion method using a computer, the health promotion method including:
acquiring identification information for identifying a person who operates an operation mechanism;
detecting a motion of the person; and
determining whether or not the detected motion satisfies a health promotion motion that is predetermined for the person identified by the identification information, in which the operation mechanism is operated in response to the operation performed by the person only when the detected motion satisfies the health promotion motion.

As described above, in the exemplary aspect, it is determined whether or not a motion of a person who operates the operation mechanism satisfies a health promotion motion that is predetermined for the person, and only when the motion of the person satisfies the health promotion motion, the operation mechanism is operated in response to an operation performed by the person. Therefore, each time the person operates the operation mechanism, the person performs the health promotion motion, and hence excellent health promotion effects can be obtained.

The motion detection unit may be a camera, and the health promotion motion may be an exercise. Excellent health promotion effects of the exercise can be obtained.

Alternatively, the motion detection unit may be a microphone, and the health promotion motion may be an utterance. Excellent health promotion effects of the utterance can be obtained.

The operation mechanism may be a lock mechanism, and the control unit may release a locked state made by the lock mechanism only when the motion of the person satisfies the health promotion motion. Each time the person releases the locked state made by the lock mechanism, the person performs the health promotion motion, and hence excellent health promotion effects can be obtained. The person may be able to select whether or not to perform the health promotion motion, and when the person selects not to perform the health promotion motion, the control unit may operate the operation mechanism without determining whether or not the health promotion motion is satisfied. In an emergency such as the occurrence of a disaster, the operation mechanism can be quickly operated without the health promotion motion being performed.

According to the present disclosure, it is possible to provide a health promotion system and a health promotion method by which excellent health promotion effects can be obtained.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a health promotion system according to a first embodiment;

FIG. 2 is a flowchart showing a health promotion method according to the first embodiment; and

FIG. 3 is a flowchart showing a health promotion method according to a second embodiment.

### DESCRIPTION OF EMBODIMENTS

Specific embodiments will be described hereinafter in detail with reference to the drawings. The same or corresponding elements are denoted by the same reference symbols throughout the drawings, and redundant descriptions will be omitted as necessary for the clarification of the description.

### First Embodiment

<Configuration of Health promotion system>

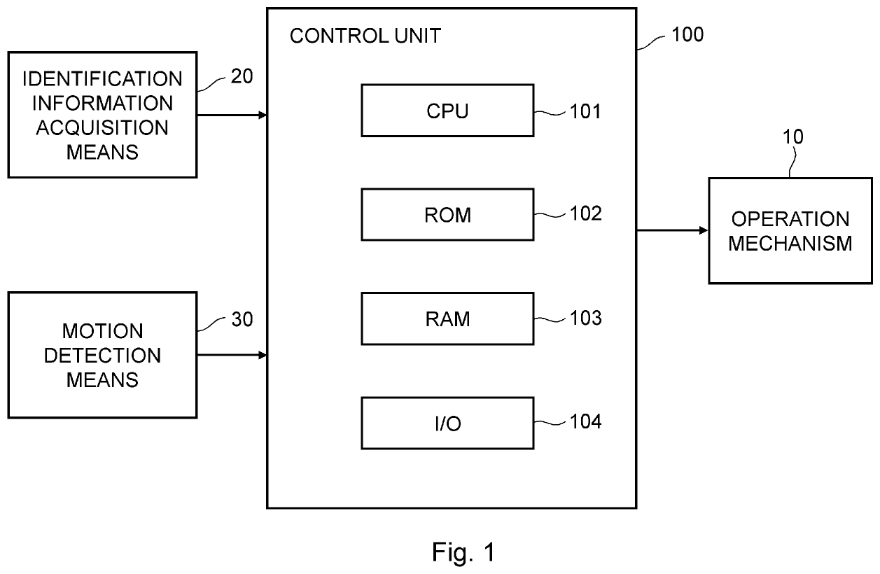

First, a health promotion system according to a first embodiment will be described with reference to FIG. 1. FIG. 1 is a block diagram showing the health promotion system according to the first embodiment. As shown in FIG. 1, the health promotion system according to this embodiment includes an operation mechanism 10, identification information acquisition unit 20, motion detection unit 30, and a control unit 100.

The entire health promotion system (i.e., the operation mechanism 10, the identification information acquisition unit 20, the motion detection unit 30, and the control unit 100) may be configured by one apparatus or by two or more apparatuses.

The operation mechanism 10 is connected to the control unit 100, which is a computer, wirelessly or by wire, and is electronically controlled by the control unit 100 and operates in response to an operation performed by a person. The operation mechanism 10 may be any mechanism that is electronically controlled by the control unit 100 and operates in response to an operation performed by a person, and the operation mechanism 10 is, for example, a lock mechanism in which a locked state is released in response to an operation performed by a person. The lock mechanism includes a physical lock mechanism which can be electronically controlled and which is provided in various types of doors etc., and also includes an electronic lock mechanism for preventing unauthorized use in an electronic device such as a smartphone and a Personal Computer (PC).

The identification information acquisition unit 20 is an electronic device which is connected to the control unit 100 wirelessly or by wire and which acquires identification information for identifying a person who operates the operation mechanism 10. The identification information acquisition unit 20 is not limited to being specific means, and is, for example, a sensor, a camera, and the like for performing biometric authentication such as fingerprint authentication, vein authentication, iris authentication, and face authentication. Further, the identification information acquisition unit 20 may be, for example, a reader that reads an Integrated Circuit (IC) card for identity verification. Further, the identification information acquisition unit 20 may be, for example, an input device such as a touch panel or a keyboard for inputting a password and the like for identity verification.

The motion detection unit 30 is an electronic device which is connected to the control unit 100 wirelessly or by wire and which detects a motion of a person who operates the operation mechanism 10. The motion detection unit 30 is not limited to being specific means, and is, for example, a camera that detects an exercise as a motion of the aforementioned person. Alternatively, the motion detection unit 30 may be a microphone or the like that detects an utterance as a motion of the aforementioned person.

Note that the identification information acquisition unit 20 may also serve as the motion detection unit 30. For example, the identification information acquisition unit 20 and the motion detection unit 30 may be the same camera. As shown in FIG. 1, the control unit 100 is connected to the operation mechanism 10, the identification information acquisition unit 20, and the motion detection unit 30 wirelessly or by wire so that it can communicate with them. The control unit 100 controls the operation mechanism 10 based on the identification information acquired by the identification information acquisition unit 20 and the motion detected by the motion detection unit 30. More specifically, the control unit 100 determines whether or not the motion detected by the motion detection unit 30 satisfies a health promotion motion that is predetermined for a person identified by the identification information acquired by the identification information acquisition unit 20. Then, the control unit 100 operates the operation mechanism 10 in response to an operation performed by a person only when the detected motion satisfies the health promotion motion.

As shown in FIG. 1, the control unit 100 includes, as hardware, a Central Processing Unit (CPU) 101, a Read Only Memory (ROM) 102, a Random Access Memory (RAM) 103, and an Input/Output (I/O) 104. That is, the control unit 100 has a function as a computer and performs various types of processing based on various types of control programs etc.

The CPU 101 is, for example, an arithmetic unit that performs control processing and arithmetic processing.

The ROM 102 is, for example, a storage unit that stores control programs and arithmetic programs executed by the CPU 101.

The RAM 103 is a storage unit that temporarily stores processing data and the like. In the RAM 103, for example, identification information of a person and a health promotion motion of the person are associated with each other and stored.

The I/O 104 is an input and output device that inputs data and signals from the outside and outputs data and signals to the outside.

Note that a health promotion motion may be any motion for promoting health, and is, for example, an exercise such as a stretching exercise, strength training, marching in place, running, jumping, and dancing. A health promotion motion is not limited to physical exercise, and may be an utterance made while a person's body is still. For example, regarding a health promotion motion for the person, the details of the motion and the number of times the person performs the motion (e.g., 10 bending and stretching exercises) are determined.

As described above, the control unit 100 determines whether or not a motion detected by the motion detection unit 30 satisfies a health promotion motion that is predetermined for a person identified by identification information acquired by the identification information acquisition unit 20. The control unit 100 determines whether or not the motion satisfies the predetermined health promotion motion based on, for example, the posture when a person does an exercise, the number of times the person does the exercise, the volume of the person's utterance, and the number of times the person says the utterance.

Then, the control unit 100 operates the operation mechanism 10 in response to an operation performed by the person only when the detected motion satisfies the health promotion motion.

That is, when a certain person operates the operation mechanism 10, the person cannot operate the operation mechanism 10 unless the person performs a health promotion motion that is predetermined for the person. Therefore, each time the person operates the operation mechanism 10, the person performs the health promotion motion, and hence excellent health promotion effects can be obtained.

As an example, the entire health promotion system according to this embodiment is configured by one smartphone. The operation mechanism 10 is, for example, a lock mechanism of the smartphone. The identification information acquisition unit 20 is, for example, a sensor or a camera built into the smartphone for performing fingerprint authentication, face authentication, or the like. The motion detection unit 30 is, for example, a camera or a microphone built into the smartphone.

Note that the identification information acquisition unit 20 and the motion detection unit 30 may be the same camera.

When an owner of a smartphone tries to release a locked state made by the lock mechanism, the control unit 100 built into the smartphone identifies the owner by identification information acquired by the identification information acquisition unit 20. Then the control unit 100 determines whether or not the owner's motion detected by the motion detection unit 30 satisfies a health promotion motion that is predetermined for the owner, and releases the locked state made by the lock mechanism only when the detected motion satisfies the health promotion motion. Therefore, each time the owner tries to release the locked state made by the lock mechanism of the smartphone, the owner performs the health promotion motion, and hence excellent health promotion effects can be obtained.

As another example, the health promotion system according to this embodiment is configured by a plurality of apparatuses. The operation mechanism 10 is, for example, a lock mechanism of a door. The identification information acquisition unit 20 is a reader that reads, for example, an IC card for identity verification. The motion detection unit 30 is, for example, a camera installed near the door.

When a certain person tries to release a locked state of the door made by the lock mechanism, the control unit 100 identifies the person by identification information acquired by the reader which is the identification information acquisition unit 20. Then the control unit 100 determines whether or not the motion of the person detected by the motion detection unit 30 satisfies a health promotion motion that is predetermined for the person, and releases the locked state of the door made by the lock mechanism only when the detected motion satisfies the health promotion motion. Therefore, each time the certain person tries to unlock the door, the person performs the health promotion motion, and hence excellent health promotion effects can be obtained.

As described above, in the health promotion system according to this embodiment, the control unit 100 determines whether or not a motion detected by the motion detection unit 30 satisfies a health promotion motion that is predetermined for a person identified by identification information acquired by the identification information acquisition unit 20. Then the control unit 100 operates the operation mechanism 10 in response to an operation performed by the person only when the detected motion satisfies the health promotion motion.

That is, a person who operates the operation mechanism 10 cannot operate the operation mechanism 10 unless the person performs a health promotion motion that is predetermined for the person.

Therefore, in the health promotion system according to this embodiment, each time the person operates the operation mechanism 10, the person performs the health promotion motion, and hence excellent health promotion effects can be obtained.

### <Health Promotion Method>

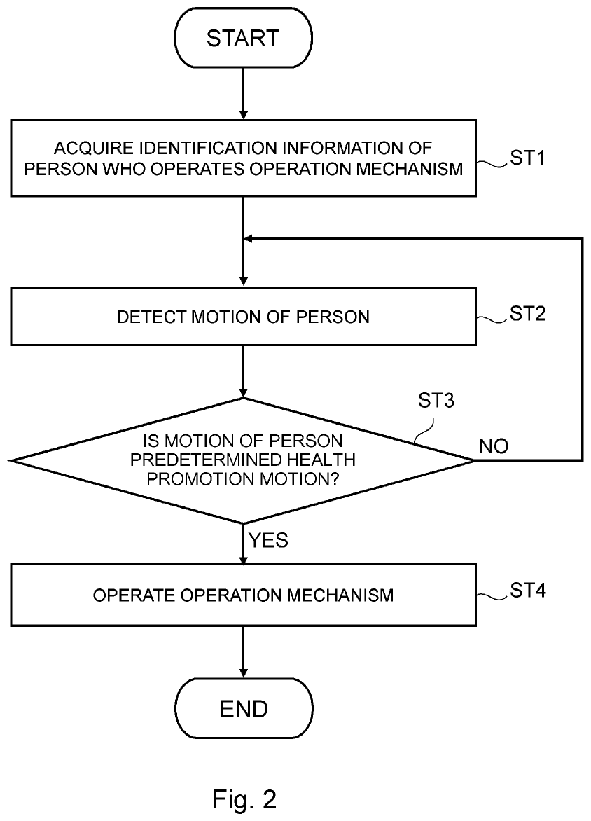

Next, a health promotion method according to this embodiment will be described with reference to FIG. 2 as well as FIG. 1. FIG. 2 is a flowchart showing the health promotion method according to the first embodiment.

First, as shown in FIG. 2, the identification information acquisition unit 20 acquires identification information for identifying a person who operates the operation mechanism 10 (Step ST1). Then the person is identified based on the identification information acquired by the identification information acquisition unit 20.

Next, as shown in FIG. 2, the motion detection unit 30 detects a motion of the person (Step ST2).

Then, as shown in FIG. 2, it is determined whether or not the motion of the person detected by the motion detection unit 30 satisfies a health promotion motion that is predetermined for the person (Step ST3). Note that the health promotion motion of the person is associated with the identification information of the person and stored, for example, in a storage unit (e.g., the RAM 103 of the control unit 100 shown in FIG. 1).

When the detected health promotion motion of the person satisfies the health promotion motion of the person (YES in Step ST3), the control unit 100 operates the operation mechanism 10 in response to an operation performed by the person (Step ST4).

On the other hand, when the detected motion of the person does not satisfy the health promotion motion of the person (NO in Step ST3), the control unit 100 does not operate the operation mechanism 10 and the process returns to Step ST2. That is, the control unit 100 does not operate the operation mechanism 10 until the detected motion of the person satisfies the health promotion motion of the person. In other words, the control unit 100 operates the operation mechanism in response to an operation performed by the person only when the detected motion of the person satisfies the health promotion motion of the person.

As described above, in the health promotion method according to this embodiment, it is determined whether or not a motion of a person who operates the operation mechanism 10 satisfies a health promotion motion that is predetermined for the person. Then, only when the motion of the person satisfies the health promotion motion, the operation mechanism 10 is operated.

That is, a person who operates the operation mechanism 10 cannot operate the operation mechanism 10 unless the person performs a health promotion motion that is predetermined for the person.

Therefore, in the health promotion method according to this embodiment, each time the person operates the operation mechanism 10, the person performs the health promotion motion, and hence excellent health promotion effects can be obtained.

### Second Embodiment

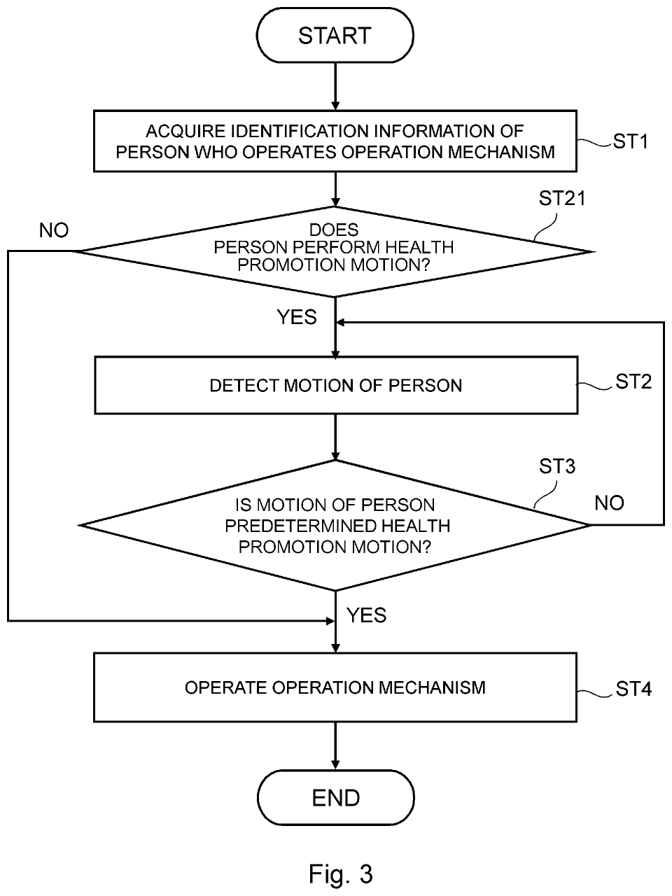

Next, a health promotion system and a health promotion method according to the second embodiment will be described with reference to FIG. 3. FIG. 3 is a flowchart showing the health promotion method according to the second embodiment. Note that Steps ST1 to ST4 shown in FIG. 3 are the same as Steps ST1 to ST4 shown in FIG. 2.

In the health promotion system according to the second embodiment, a person who operates the operation mechanism 10 can select whether or not to perform a health promotion motion.

Therefore, as shown in FIG. 3, after the identification information acquisition unit 20 acquires identification information for identifying a person who operates the operation mechanism 10 (Step ST1), the person selects whether or not to perform a health promotion motion (Step ST21).

When the person selects to perform a health promotion motion (YES in Step ST21), the control unit 100 performs Steps ST2 to ST4 like in the case of FIG. 2.

On the other hand, when the person selects not to perform a health promotion motion (NO in Step ST21), the control unit 100 operates the operation mechanism 10 in response to an operation performed by the person without performing Steps ST2 and ST3 (Step ST4).

As described above, in the health promotion method according to this embodiment, a person who operates the operation mechanism 10 can select whether or not to perform a health promotion motion.

Since a person who operates the operation mechanism 10 usually selects to perform a health promotion motion, excellent health promotion effects can be obtained like in the health promotion method according to the first embodiment.

On the other hand, for example, when the operation mechanism 10 is a lock mechanism of a door or a smartphone, a locked state made by the lock mechanism can be quickly released in an emergency such as the occurrence of a disaster by selecting not to perform a health promotion motion.

The configurations other than the above ones are similar to those of the first embodiment, and the descriptions thereof will thus be omitted.

In the above example, the program includes instructions (or software codes) that, when loaded into a computer, cause the computer to perform one or more of the functions described in the example embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health promotion system using a computer, the health promotion system comprising:
an operation mechanism configured to operate in response to an operation performed by a person;
a control unit configured to control the operation mechanism;
identification information acquisition unit that acquires identification information for identifying the person who operates the operation mechanism; and
motion detection unit that detects a motion of the person, wherein
the control unit determines whether or not the motion detected by the motion detection unit satisfies a health promotion motion that is predetermined for the person identified by the identification information, and
the control unit operates the operation mechanism in response to the operation performed by the person only when the detected motion satisfies the health promotion motion.

2. The health promotion system according to claim 1, wherein the motion detection unit is a camera, and the health promotion motion is an exercise.

3. The health promotion system according to claim 1, wherein the motion detection unit is a microphone, and the health promotion motion is an utterance.

4. The health promotion system according to claim 1, wherein
the operation mechanism is a lock mechanism, and
the control unit releases a locked state made by the lock mechanism only when the motion of the person satisfies the health promotion motion.

5. The health promotion system according to claim 1, wherein
the person is able to select whether or not to perform the health promotion motion, and
when the person selects not to perform the health promotion motion, the control unit operates the operation mechanism without determining whether or not the health promotion motion is satisfied.

6. A health promotion method using a computer, the health promotion method comprising:
acquiring identification information for identifying a person who operates an operation mechanism;
detecting a motion of the person; and
determining whether or not the detected motion satisfies a health promotion motion that is predetermined for the person identified by the identification information,
wherein the operation mechanism is operated in response to the operation performed by the person only when the detected motion satisfies the health promotion motion.

7. The health promotion method according to claim 6, wherein the motion of the person is detected by a camera, and the health promotion motion is an exercise.

8. The health promotion method according to claim 6, wherein the motion of the person is detected by a microphone, and the health promotion motion is an utterance.

9. The health promotion method according to claim 6, wherein
the operation mechanism is a lock mechanism, and
a locked state made by the lock mechanism is released only when the motion of the person satisfies the health promotion motion.

10. The health promotion method according to claim 6, wherein
the person is able to select whether or not to perform the health promotion motion, and
when the person selects not to perform the health promotion motion, the operation mechanism is operated without determining whether or not the health promotion motion is satisfied.
